(19) United States
(12) Patent Application Publication
(10) Pub. No.: US 2026/0234719 A1
(43) Pub. Date: Aug. 13, 2026

(54) HIGH NUCLEIC ACID SEQUENCE DISCRIMINATION USING A MULTIFACETED NUCLEIC ACID MOLECULE

(71) Applicant: ASECUD SA, Lausanne (CH)

(72) Inventors: Amar RIDA, Chavannes-près-Renens (CH); Vera Monica LEMOS DA SILVA, Cossonay-Ville (CH)

(21) Appl. No.: 19/156,920

(22) PCT Filed: Feb. 16, 2024
(86) PCT No.: PCT/EP2024/054059

§ 371 Date: Aug. 15, 2025

(30) Foreign Application Priority Data
Feb. 16, 2023 (CH) 0001462023

(51) Int. Cl. C12Q 1/6876; C12Q 1/6853

(52) U.S. Cl. CPC C12Q 1/6876; C12Q 1/6853; C12Q 2600/156

(57) ABSTRACT

A complex nucleic acid molecule for binding to a region of a target nucleic acid sequence comprising a) a first single-stranded nucleic acid sequence, having a 5′ terminus and a 3′ terminus, and being complementary at least in part to the target sequence; wherein the said complementary portion comprises a priming region and a hooking region positioned respectively at the 5′ and 3′ terminus sides of the said first single-stranded nucleic acid sequence; b) a second blocking single-stranded nucleic acid sequence, having a 5′ terminus and a 3′ terminus, which is at least in portion complementary to the hooking region of the first single-stranded nucleic acid sequence and c) a linker region connecting the 5′ terminus of the first single-stranded nucleic acid sequence to the 3′ terminus of the second single-stranded nucleic acid sequence, wherein the linker comprises at least one non-nucleic acid chemical group. In a preferred embodiment the said nucleic acid molecule is further designed to have an oligonucleotide label function which leads to the generation of a detectable signal allowing the detection of the target nucleic acid sequence.

### FIELD OF THE INVENTION

The invention generally relates to compositions and structures of oligonucleotide molecules capable of specifically binding to a target nucleic acid sequence and the methods of their use for the detection, identification, and/or quantification of target nucleic acids in a sample.

### BACKGROUND OF THE INVENTION

Short nucleic acid sequences have been used for a long time to recognize and bind target complementary sequences. This allows nucleic acid identification and is thus used as a technique with applications in gene expression, organism identification, labelling, among others. One of the most broadly used techniques using short nucleic acid sequences, called primers, is Polymerase Chain Reaction (PCR) where primers recognize a sequence, bind, and extend it by incubation with the DNA Polymerase, allowing the detection of its presence. This target-amplification technique has nowadays applications in in vitro diagnostics ranging from Infectiology, Forensics, Genetics, Oncology, etc. Many such applications require specific, sensitive, and inexpensive quantitative detection of nucleic acid sequences. While the performance of current PCR techniques is constantly improving, there are still many challenges that have not been fully overcome to render this technique a golden conventional for in vitro diagnostics.

There are two main issues when using short nucleic acid sequences in a reaction: primer-dimers and mispriming. Primer-dimers occurs when 2 primers hybridize to each other by complementary overhangs on the 3′ terminal side leading to extension and amplification. This in turn can lead to false positive signals as well as depletion of cofactors needed for the amplification of the intended targets reducing the robustness and sensitivity of the assay. Mispriming occurs when the primer binds to another target that is only partially complementary to the primer, decreasing assay specificity and overall sensitivity.

From the application side, nucleic acid sequences-driven amplification and detection methods are generally used for the detection of pathological conditions. However, these pathological conditions sometimes differ from the “normal” healthy conditions by one single nucleotide. Accordingly, distinguishing allelic variants such as single nucleotide polymorphisms (SNPs) is a typical example of application for target-amplification methods. Conventionally used primers, “linear” with typical size of 20 nucleotide long sequences, do not effectively distinguish two sequences that differ from each other by one single nucleotide substitution. This issue becomes even more pronounced, if the targeted single nucleotide different sequences are diluted within a mixture containing more abundant “mismatched” sequences.

One way to approach this is to include the variation in the sequence within the product of amplification (“amplicon”) by placing primers around that region and detecting the variation using specific probes. Such technique, however, has a sensitivity that does not go below 3% of targeted matched sequences within a mismatched abundant sequence background due to the overwhelming amplification of the prevalent mismatched sequences (Tyagi et al., 1998, Nature Biotechnology 16:49-53). To detect variant sequences, especially if they are rare, one must have very selective primers that bind specifically to the sequence being investigated, the amplicon being then detected by DNA intercalating dyes, labelled primers or probes that do not differentiate between variant sequences. An important issue with this is that a primer to be very specific needs to be of a certain length (18-30 nucleotides). Long primers are not very selective since a mismatch would not greatly interfere with its binding. Therefore, to be very selective, primers must be of short length, and these tend to have a very low melting temperature being unlikely to hybridize with target sequences at typical reaction temperatures.

One approach to make use of primers being selective while retaining specificity is Amplification Refractory Mutation System (ARMS). An ARMS primer has a 3′ terminal nucleotide that is complementary to the targeted sequenced, being a mismatch to the other variant allele (Newton et al., 1989, Nucleic Acids Res. 17:2503-2516; Ferrie et al., 1992, Am. J. Hum. Genet. 51:251-262). This technique relies on the property of certain DNA polymerases to avoid extension of primer-target hybrids having a mismatch on the 3′ terminal side leading to a delay of the amplification of the mismatched sequence. ARMS has been shown useful for determining zygosity (homozygous for one allele or heterozygous) where the amount of the intended target is relatively high but for low quantities of the targeted sequence, this technique has a detection limit of about 1%.

Making use of the idea of gathering the properties of long primers for specificity and short primers for selectivity, Jong-Yoon Chun and his colleagues at the Seegene Institute of Life Science in Seoul, South Korea, have designed the “dual-priming oligonucleotide (DPO)” (Chun et al. (2007) Nucleic 50 Acids Res. 35 (6) e40). This DPO is comprised of 3 separate segments: one 5′ with high melting temperature, a central region not complementary to the target composed of deoxyriboinosines and a 3′ shorter sequence with low melting temperature. While the 5′ region confers specificity to the target, the 3′ shorter sequence confers selectivity being fully complementary to the targeted sequences. Only when both complementary sections (5′ and 3′) are bound to the target, the primer is extended. The need to contain a “weakly complementary” region based in deoxyriboinosines to separate the 2 regions responsible for the intended functions (selectivity and specificity) restricts the optimal working temperature ranges, where low annealing temperatures allow the deoxyriboinosines to bind weakly and not form a real separation. Nevertheless, the usage of deoxyriboinosines in the primers makes sure that selection of the intended target sequence is done in every cycle, although at the expense of assay performance and sensitivity.

To increase annealing specificity, the U.S. Pat. No. 8,440,406 disclosed a design called “Iso-base Amplification Primer (IAP)” that contains an iso-region separating 3′ and 5′ segments (USB2). This iso-region includes at least two contiguous or non-contiguous non-conventional bases, such as iso-C and/or iso-G. This so-called iso-region in combination with what they called annealing factors (primer length, pH, annealing temperature, pH, and secondary structures) dictate the specificity of the primer. “IAPs” have increased sensitivity in subsequent amplification cycles compared to conventional primers since iso-bases pair more favourably with their complementary iso-base compared to any other base or base substitution.

To improve the sensitivity, herein termed as the ability to detect rare targeted sequences in the presence of abundant sequences differing by few or a single nucleotide, Vladimir Makarov, and his colleagues described in International Patent application WO 2012/112582 a design of a “discontinuous polynucleotide primer”. This polynucleotide primer is composed of 2 separate primers, one long (“fixer”) and one short (“primer”), connected by a long high-temperature shared stem formed by the 3′ tailed sequence of the long primer and the 5′ tail sequence of the short primer. The “fixer” is sufficiently long (about 30 nucleotides) to provide stable annealing and allowing the “primer” to effectively select targeted sequences with high PCR efficiency. Further improvements are portrayed in Example 10 of WO 2012/112582 A2, including an LNA on the 3′ terminal nucleotide of the “primer” as in ARMS, further increasing selectivity. Although, the limit of detection of this “polynucleotide primer” is as low as 0.01% (one mutant allele in 14,000 wild-type alleles), the primer is very long and heavy compromising PCR efficiency. Moreover, in the design of such long primer sections, especially in a multiplexing reaction, it would be nearly impossible to avoid unspecific interactions between all the different regions. Further improvements are disclosed in FIGS. 12 and 13 of WO 2012/112582 where modifications to the “fixer” and the “primer” render them more specific when binding to the target sequence. These modifications include a stem-loop structure (single stranded poly-dT).

To further improve sensitivity as well as selectivity, highly selective primers termed “SuperSelective” Primers were disclosed by Salvatore Marras and colleagues (European patent No 3,346,016, Vargas et al. J Mol Diagnostics, 2022, Vol 24, No. 3). As for the “DPO” primers, “SuperSelective” Primers are composed of 3 regions: a long 5′ anchor sequence to confer specificity to the primer, a short 3′ foot sequence conferring selectivity and a middle bridge sequence allowing the formation of one big loop by not being complementary to the with the “intervening sequence”. Accordingly, the bridge sequence divides the 2 regions as in the DPO without the usage of deoxyriboinosines, comprising instead a relatively long nucleotide sequence segment that is not complementary to the target sequence and it does not form secondary structures, forming a “bubble”. The 3′ “foot sequence” includes the “interrogating nucleotide” which is complementary to the targeted sequence mismatching the other variant sequence. As opposed to the DPO, the bridge sequence will be incorporated into the amplified regions thus increasing the efficiency of the PCR in subsequent cycles, leaving the first cycles as pivotal for the selectivity of the assay. For the mode of action of “SuperSelective” Primers, it is crucial that the bridge is long to create a “bubble” large enough to destabilize the foot region guaranteeing selectivity towards targeted sequence. These features while displaying high sensitivity (0.01%, 10 copies of rare mutant target sequence within a mixture containing 100,000 copies of closely related wild-type sequences) are very sequence dependent to achieve desired selectivity and sensitivity.

To improve the primer specificity and reduce primer—dimers, U.S. Pat. Nos. 6,365,729 and 6,277,607, disclose the use of an oligonucleotide primer having a hairpin structure (also known in the art by loopable oligonucleotide structure) for nucleic acid amplification including extension of primers by a DNA polymerase. The primers include a type of hairpin structure in which a single-stranded loop separates complementary 3′ and 5′ arms and in which the loop and the 3′ arm are complementary to the target nucleic acid. Based on a similar design, the European patent No. 3,737,773 disclosure utilizes a loopable oligonucleotide primer. This design features a loop containing molecular indexing and adaptor sequences for barcoding target nucleic acids, allowing identification through next-generation sequencing. Notably, the patent highlights the benefit of the closed 3′ arm in this loopable design. It helps reduce bias caused by unintended primer interactions during high-multiplexing target amplification by PCR. Within the same spirit, the US patent application No. 2021/0054369, discloses the use of a hairpin primer design to produce targeted sequencing libraries.

The design of the primers mentioned above are all sequence dependent therefore possessing variable efficiency depending on the intended specific target. This is stated by Vargas et al (J Mol Diagnostics, 2022, Vol 24, No. 3) where an A-T rich foot region as found on BRAF V600E primers discriminates more than a G-C one found on EGFR L858R primers. Moreover, all of these, including conventional “linear” primers, “ARMS” primers, “DPO”, “IAP”, “Polynucleotide Primer”, “SuperSelective” primers are prone to generating false-positive signals or reduce assay sensitivity in a high multiplexing reaction due to their free 3′ ends. These can be bound by small partial complementary sequences and initiate the exponential synthesis of unintended amplicons, even in the absence of perfectly complementary target sequences. Vargas et al. demonstrated a multiplex “SuperSelective” assay with a 9-plex PCR reaction and detected 10 copies of the targeted mutant sequences, although it relies on the specificity of the of specificity of molecular beacons probes (J Mol Diagnostics, 2022, Vol 24, No. 3), the number of targets in the multiplexing reaction will be limited to further since the short 3′ “foot sequence” is free, and it will exponentially be more prone to have partial complementary sequences as one increases the number of targets in the same reaction.

The use of hairpin (loopable) primers as stated above, offer a promising approach for PCR by preventing free 3′ termini interactions and thereby limits the primer extension by DNA polymerizes only in case where the specific loop sequence is bound to its target sequence. However, existing designs of loopable primers (as disclosed for instance in U.S. Pat. Nos. 6,365,729 and 6,277,607) suffer from reduced amplification efficiency due to the closed hairpin structure, hindering the relatively short loop sequence (<30 nucleotides) from effectively binding to its target and have practical detection limits also of about 1%. Additionally, incorporating large non-specific sequences, like barcodes (as disclosed in the European patent No. 3,737,773), introduces further non-specific interactions, limiting their applicability.

The applicable known procedures and approaches have shortcomings, including the requirement and constraints for flexible oligonucleotide probe design allowing easy detection of targeted nucleic acids sequences with high specificity and sensitivity under high multiplexing conditions. Furthermore, the current disclosure further stresses the need for an oligonucleotide probe that allow the detection of diverse, rare genetic variants (less than 1%) like mutations amidst a mixed population of cells or wild-type nucleic acids.

### SUMMARY OF THE INVENTION

The present invention introduces a flexible oligonucleotide probe design, termed “ReBound” Primer (RB Primer). The disclosed probe enables: (1) Efficient and selective binding to a target nucleic acid sequence, (2) High sensitivity, detecting low copies of the targeted allelic variants amidst 1 million copies of wild-type alleles, (3) Reduced artifacts and minimized non-specific interactions, particularly during high-multiplexing amplification, (3) Effective probing and labelling target sequences in various molecular techniques, including real-time PCR and next-generation sequencing.

Accordingly, the invention herein discloses an oligonucleotide molecule for binding to a target nucleic acid of the FIG. 1 that comprises:
a) a first single-stranded nucleic acid sequence (1, 2), having a 5′ terminus and a 3′ terminus, and being complementary at least in part to the target sequence (5) with a length of 15 to 40 nucleotides.
b) The said complementary portion of the first single-stranded nucleic acid sequence comprises a priming region (1) and a hooking region (2) positioned respectively at the 5′ and 3′ terminus sides.
c) a second blocking single-stranded nucleic acid sequence (4), having a 5′ terminus and a 3′ terminus, which is at least in portion complementary to the hooking region of the first single-stranded nucleic acid sequence.
d) a linker region (3) connecting the 5′ terminus of the first single-stranded nucleic acid sequence (1) to the 3′ terminus of the second single-stranded nucleic acid sequence (2), wherein the said linker (3) has no more than five contiguous bases that are complement to first nucleic acid sequence (1,2) or to the target nucleic acid sequence.

Accordingly, the hooking region (2) of the disclosed oligonucleotide molecule is shorter (between 4 to 15 nucleotides) than the priming region (1) and at least partly complementary to the blocking region (4) resulting thereby into an oligonucleotide molecule having a loopable (hairpin like) structure. This last feature allows to maintain the hooking region (2) sequence with the 3′ terminus in close proximity with the blocking region sequence (4), competing thereby with potential undesirable interactions of the 3′ terminus of the hook with other nucleic acid molecules in the same reaction.

Another feature of the oligonucleotide molecule of the present invention is that it includes a linker (3) between the priming region sequence (1) and the blocking region (4). This linker exhibits minimal or no interactions with surrounding nucleic acid sequences and, preferably, no interactions with naturally occurring nucleic acids. With this respect, the linker is preferably composed from at one non-nucleic acid chemical group. In a preferred embodiment the linker is a polymer composed from non-nucleic acid chemical groups.

Additionally, it is designed to have a linker length that confer a flexibility to the molecule, ensuring thereby that the binding of the priming region (1) to the target sequence (5) is substantially as effective as if the sequence were linear (not forming a closed loop structure).

Accordingly, the disclosed composition of the oligonucleotide molecule of the invention provides it with a steric conformation that is less constrained, resulting in a binding and amplification efficiency that are closer to an open linear primer.

In a preferred embodiment according to the invention, the linker is composed of at least one Polyethylene glycol polymer such as poly (hexaethylene glycol) H(OCH₂CH₂)₆ₙOH, with n varies between 1 and 18 and preferably between 1 and 5.

In a preferred embodiment according to the invention, the length of the linker (3) is between ¼ to 5/4 the length of the priming region (1). Preferably the length of the linker is substantially equal to the length of the priming region (1).

In a preferred embodiment according to the invention, the oligonucleotide molecule disclosed herein is used to amplify the target nucleic acids sequence (5) enzymatic amplification using techniques like PCR. This would lead to amplification of the target sequence with a similar performance as a conventional linear nucleic acid primer formed by the first single-stranded nucleic acid sequence (1, 2).

In a preferred embodiment according to the invention, the oligonucleotide molecule further comprises a label fluorescence resonance energy transfer (FRET) label system that includes a fluorescent reporter molecule (9) and a quencher molecule (8). The said fluorescent reporter molecule (9) and a quencher molecule (8) are located on the blocking single-stranded nucleic acid sequence (4). In an alternative embodiment, the fluorescent reporter molecule (9) and a quencher molecule (8) are separately located on the blocking single-stranded nucleic acid sequence (4) and the hooking region (2).

In a preferred embodiment according to the invention, the oligonucleotide molecule further comprises a spacing region (11) of 5-20 nucleotides between the priming (1) and hooking (2) regions, with no complementarity to the target sequence (5). This gap between the priming (1) and hook (2) regions creates two segments with different thermodynamic binding characteristics to their respective target sequence (5): a more stable 5′ priming region with a higher melting temperature

$$( {T}_{m}^{R} )$$

and a less stable 3′ hook region with a lower melting temperature

$$( {T}_{m}^{H} ) .$$

This design makes the hook binding to its target sequence (5) to be in competition for binding with the blocking region (4). This competition is the key feature that makes the binding of the hooking sequence to its complement target sequence very sensitive to potential mismatches between the two sequences (target and the hook). Therefore, this sensitivity to mismatches at the hook level is the basis for making the ReBond primer discriminative toward the single nucleotide variants within the target sequence.

To detect nucleotide variants at a single nucleotide resolution level, the hook region sequence (2) of the oligonucleotide molecule of the present invention needs to perfectly match the target nucleic acid at the mutated nucleotides positions. This match creates a stronger binding affinity of the hook toward its target mutated sequence. This stronger bond is characterized by a melting temperature

$$( {T}_{m}^{HM} )$$

that is higher compared to the melting temperature

$$( {T}_{m}^{HW} )$$

when the hook mismatches the wild-type sequence. Within this design, the spacing sequence (11) accentuates the instability of the interaction between the hook and the target sequence, further increasing the difference in melting temperatures between the mutated

$$( {T}_{m}^{HM} )$$

and wild type

$$( {T}_{m}^{HW} )$$

sequences. Additionally, the blocking region (4), that's kept in proximity by the linker (3), come into play to further compete with the binding of the hook to its target sequence (5). This competition will further discriminate the binding of the hook to the mashed mutated and unmatched wild-type sequencing. This discrimination will be optimal under conditions where:

$${T}_{m}^{HW} < {T}_{m}^{RB} < {T}_{m}^{HM} , wherein {T}_{m}^{RB}$$

is the melting temperature of the ReBound primer oligonucleotide and that characterizes the biding strength of the hook to the blocking regions.

Accordingly, the invention herein discloses an oligonucleotide for detecting a first allelic variant of a target nucleic acid sequence in a sample suspected of comprising at least a second allelic variant of the said target nucleic acid sequence, comprising:
(a) a first single-stranded nucleic acid sequence (1, 2), having a 5′ terminus and a 3′ terminus, and being complementary at least in part to the target sequence (5).
(b) The said complementary portion of the first single-stranded nucleic acid sequence comprises a priming:
   i. a priming region (1), with a length between 15 to 40 nucleotides, located at the 5′ terminus of the said first single-stranded nucleic acid sequence and that bind indiscriminately to both first and second allelic variants.
   ii. a hooking region (2), with a length between 4 to 15 nucleotides, located at the 3′ terminus of the said first single-stranded nucleic acid sequence and that bind discriminately with a higher affinity to a first allelic variant compared to the second allelic variant.
   iii. a spacing region sequence (11) which is not complementary to the target sequence (5), with a length between 5 to 10 nucleotides, located between the priming (1) and the hooking (2) regions.
(c) a second blocking single-stranded nucleic acid sequence (4), having a 5′ terminus and a 3′ terminus, which is at least in portion complementary to the hooking region of the first single-stranded nucleic acid sequence.
(d) a linker region (3) connecting the 5′ terminus of the first single-stranded nucleic acid sequence (1) to the 3′ terminus of the second single-stranded nucleic acid sequence (2), wherein the said linker (3) has no has no more than five contiguous bases that are complement to first nucleic acid sequence (1,2) or to the target nucleic acid sequence.

Furthermore, in a preferred embodiment the spacing region (11) which is not complementary to the target sequence so it can thus be used as a unique identifier of that specific reaction since it can be incorporated into the extended strand by an enzymatic amplification reaction. In fact, the incorporation of the spacing region into the extended strand will be amplified as it serves as a template to the antiparallel strand amplification.

In another preferred embodiment according to the invention, the oligonucleotide molecule of the invention, further comprises an extension sequence (16) positioned at its 5′ terminus of the blocking sequence (4) and characterized by not being: (a) complementary to the hooking region (2), (b) an active template for the extension of the hooking region (2), while bound to the blocking sequence (4), by a sequence polymerization reaction. In a preferred embodiment, the said characteristic (b) is achieved by having a mismatch, positioned at the end of 3′ terminus of the hooking region (2), between the blocking sequence (4) and the hooking region (2). In another preferred embodiment, the said characteristic (b) is achieved by having a sequence polymerization reaction blocker (13) positioned at the 5′ terminus of the blocking sequence (4). In practice, the extension sequence (16) positioned at its 5′ terminus of the blocking sequence (4) is designed to be complementary and bind to the antiparallel sequence (14) resulted from the extension of the hooking region (2) upon its binding to the target nucleic acid (5) by a sequence polymerization reaction. This feature will be practically useful for the detection of larger insertion and deletion variants.

The present invention further discloses a method for selectively detecting a target nucleic acid sequence in a sample, comprising the use of a primer oligonucleotide molecule for binding to a region of the said target nucleic acid with the composition described above.

The present invention further discloses a kit for detecting at least one target nucleic acid in a sample comprising at least one oligonucleotide molecule for binding to a region of the said target nucleic acid with the composition described above.

Other applications for this invention will occur to persons skilled in the art. Different embodiments are set out in the dependent claims. The subject matter of the claims and all claimed combinations is incorporated by reference in this description and remains part of the disclosure event if claims are abandoned.

Further, those skilled in the art will appreciate that various adaptations and modifications of the just-described preferred embodiments can be configured without departing from the scope and spirit of the invention. Therefore, it is to be understood that, within the scope of the appended claims, the invention may be practiced otherwise than as specifically described herein.

### BRIEF DESCRIPTION OF THE DRAWINGS

The objects and features of the present invention are set forth with particularity in the appended claims. The present invention, both as to its composition and manner of operation, together with further objects and advantages, may best be understood by reference to the above description, taken in connection with the accompanying drawings, wherein:

FIG. 1 is a schematic representation of the oligonucleotide molecule depicting its main different parts disclosed herein. All the different components are portrayed, including the hooking, blocking, and spacing regions, as well as the linker and fluorescent/quencher labels. The features leading to an intrinsic detection method and the blockage of non-specific interactions are highlighted. This version of the oligonucleotide molecule as disclosed herein is called “Labelled Discriminative ReBound” Primer (LDRB).

FIG. 2 is a schematic representation of the oligonucleotide molecule depicting its main different parts disclosed herein. This oligonucleotide molecule further includes the blocker sequence that is an extension of the blocking sequence. The said blocker sequence is complementary to a specific variant of the enzymatically extended sequence of the target nucleic caid. This version of the oligonucleotide molecule as disclosed herein is called “Labelled Blocking ReBound” Primer (LBRB).

FIG. 3: (a) is a schematic representation of the basic design of the oligonucleotide ReBound primer (RB) of the invention, which comprise a priming region (1), the hook region (2), the blocking region (4) and the linker (3). (b) is a schematic representation of the ReBound Primer when hybridized to its intended target sequence (5) leading to 3′ terminus extension (6) by an enzymatic amplification reaction.

FIG. 4 is a schematic representation of the ReBound primer displaying its main different parts including a spacing region (11). (b) is a schematic representation of the ReBound primer with a spacing sequence that hybridize to its intended target sequence (5) leading to 3′ terminus extension (6) by an enzymatic amplification reaction. This version of the ReBound Primer that includes that spacer as disclosed herein is termed “Discriminative ReBound” Primer (DRB).

FIG. 5 is a schematic representation of the DRB: (a) hybridized to its intended target sequence (5) leading to 3′ terminus extension (6). (b) The extended sequence incorporates the spacing sequence (11) that will then serve as a template for the reverse extension (6′) of the reverse primer (10). (d) The resultant double-stranded amplicon will thus incorporate a unique identifier sequence (11) that is specific to the used DRB.

FIG. 6 is a schematic representation of the DRB: (a) hybridized to a target sequence (5) when the hooking region encounters a mismatch (12). (b) The mismatch leads to weaker binding of the hooking region (2) to target (wild-type) variant sequence, favoring thereby the competing binding of the hooking region (2) to the blocking region (4).

FIG. 7 is a schematic representation of the representation of the DRB where the spacing region (11) is at least partly complementary to the blocking region (4).

FIG. 8 is a schematic representation of a ReBound primer embodiment, which further includes an extended region (7) of the blocking region (4), which further comprises a FRET label (8,9). The presence of a blocker (13) between blocking region (4) and extended region (7) prevents the 3′ terminus enzymatic extension of the hooking region (2) when it's bound to the blocking region (4).

FIG. 9 is a schematic representation of a ReBound primer embodiment of FIG. 8, which further includes a spacing region (11).

FIG. 10 is a schematic representation of a ReBound primer embodiment of FIG. 9, with its mode of action when hybridized to its intended target sequence: (a) The binding of the ReBound primer to its target sequence leads to 3′ terminus extension (6) using enzymatic amplification reaction. (b) The extension (6′) of an antiparallel primer (10) leads to the cleavage and release of the fluorescent label (9) that was bound to the extended sequence (14).

FIG. 11 is a schematic representation of another embodiment of a labelled ReBound Primers that includes a FRET label (9,8) located on the hooking and the blocking regions.

FIG. 12 shows the comparative results for PCR linearity and performance between conventional “linear”, “hairpin” and “RB” Primers using a dilution series of targeted template (a) as well as the mean detected Ct values for 1000, 100, 10 copies of template for each primer design (b) from Example 1.

FIG. 13 depicts the sensitivity to a single mismatch in the target between conventional “linear”, “hairpin” and “RB” Primers as detected by PCR amplification from Example 2.

FIG. 14 demonstrates the property of the “RB” Primer to avoid interactions with partially complementary 3′ sequences in both cross-dimers (a) and self-dimers (b) in comparison to conventional “linear” and “hairpin” primers. Rectangle emphasizes the presence of self-dimers on the melting curve analysis from Example 3.

FIG. 15 is an example of the multiplexing capability of “RB” primers reducing non-specific signal from Example 4.

FIG. 16 portrays an example of a DRB design in comparison with the conventional “linear” and “hairpin” primers. (a) and (b) show the linearity of both controls and DRB primers identifying the delay introduced by the usage of a “hairpin” primer. (c) and (d) demonstrate the single mismatch discrimination for the same amount of template between controls and DRB primers from Example 5.

FIG. 17 exhibits another example of the linearity and performance as well as single mismatch discrimination of one “DRB” Primer from Example 6. Linearity and PCR efficiency are very high for the “DRB” primer as seen in the dilution series of the matched template (a). The selectivity of the “DRB” Primer discriminating a single mismatch by 22 Cₜ value difference is shown in b). Sensitivity of “DRB” Primer is set at 0.001% since 10 copies of mutant are discriminated from a 1,000,000 mismatched sequences background (c).

FIG. 18 shows the results obtained in Example 7 when using an LDRB as shown in FIG. 9. (a) LDRB is an efficient primer as seen by high linearity in PCR using a dilution series of the matched template. (b) LDRB further increases single mismatch discrimination as seen by a further delay in amplification of the mismatched sequence in comparison to the same design without the fluorophore and quencher on the blocking region.

FIG. 19 is a schematic representation of the the ReBound primer displaying its main different parts additionally to an extended blocking sequence (16) and an amplification Blocker (13). This version of the Rebound Primer is called “Blocking ReBound Primer (BRB)”.

FIG. 20 is a schematic representation of the BRB primer mode of action when hybridized to its intended target sequence: (a) after binding and extension (6) of the BRB Primer, the extended sequence (14) will be fully complementary to the blocker sequence (16) which will then halt extension of the antiparallel strand blocking the amplification reaction; (b) the extended sequence (14) contains an insertion and/or deletion (15) which will then not match at least partially to the blocker sequence (16) hence not being able to halt the extension of the antiparallel strand and leading to an amplification reaction.

### DETAILED DESCRIPTION OF THE INVENTION

This invention is based on a unique and flexible design of an oligonucleotide molecule acid molecule whose different portions may be adapted to better suit the intended applications.

Accordingly, the present invention discloses an oligonucleotide molecule for binding to a target nucleic acid molecule within a sample that comprises a first single-stranded nucleic acid sequence (1, 2), having a 5′ terminus and a 3′ terminus, and being complementary at least in part to the target sequence (5). This first nucleic acid sequence is composed from two regions: (a) a long first single-stranded priming nucleic acid sequence (1), with a length of 15 to 40 nucleotides, positioned at the 5′ of the said first nucleic acid sequence and that aims to trigger the binding of the said oligonucleotide molecule to the target nucleic acid molecule; (b) a shorter hooking region (between 4 to 15 nucleotides long) positioned at the 3′ of the said first nucleic acid sequence and that upon its binding to the target nucleic acid will initiate an extension and amplification of the said target nucleic acid by an enzymatic polymerization reaction.

Furthermore, the oligonucleotide molecule of the invention includes a second blocking single-stranded nucleic acid sequence (4), having a 5′ terminus and a 3′ terminus, which is at least in portion complementary to the hooking region of the first single-stranded nucleic acid sequence. The 3′ terminus of the blocking single-stranded nucleic acid sequence (4) is connected to the 5′ terminus of the priming region (1) via a linker region (3).

The linker (3) is characterized by: (a) having a length that's between ¼ to 5/4 the length of priming region and preferably between substantially equal to the length of priming region; (b) it has no more than five contiguous bases that are complement to first nucleic acid sequence (1,2) or to the target nucleic acid sequence. To reach in an easier way the last design objective of having low complementary between the linker and the “surrounding nucleic acids sequences”, the linker is preferably made from components with no interactions with natural nucleic acids. With this respect, the linker is preferably composed from at one non-nucleic acid chemical group. In a preferred embodiment the linker is a polymer composed from non-nucleic acid chemical groups.

Accordingly, the present invention discloses an oligonucleotide having a loopable (hairpin like) structure. This last feature allows to maintain the hooking region (2) sequence with the 3′ terminus in close proximity with the blocking region sequence (4), competing thereby with potential undesirable interactions between the 3′ terminus.

Another feature of the oligonucleotide molecule of the present invention is the length of the linker (3) that is set to confer a flexibility to the molecule, ensuring thereby that the binding of the priming region (1) to the target sequence (5) is substantially as effective as if the sequence were linear (not forming a closed loop structure).

Accordingly, the disclosed composition of the oligonucleotide molecule of the invention provides it with a steric conformation that is less constrained, resulting in a binding and amplification efficiency that are closer to an open linear primer. Furthermore, oligonucleotide molecule of the invention presents low undesirable interactions with the surrounding nucleic acids in the sample. This is achieved first by having the blocking competing with non-specific interactions of 3′ terminus of the hook and the surrounding free nucleic acids. Second, the non-specific interactions are further limited by having a linker with minimal to no interactions with surrounding nucleic acid sequences and, preferably, exhibits no interactions with naturally occurring nucleic acids.

The oligonucleotide nucleic acid molecule disclosed herein can be used in different applications that are dependent on the binding/recognition of the target nucleic acid sequence. These include, but are not limited to, PCR, digital PCR, Reverse Transcription, DNA sequencing, RNA sequencing, Isothermal amplification, Fluorescence in Situ Hybridization (FISH), Site-directed mutagenesis, among others. The target nucleic acid sequence may be any sequence of interest: DNA (e.g. genomic DNA), RNA (e.g., mRNA), and/or cDNA. A sample may be any sample that contains, or is suspected of containing nucleic acids, either after purification (extraction of nucleic acids) or in crude form any type of biological samples after the appropriate sample preparation techniques known in the art.

As disclosed before, the key primary feature of the oligonucleotide nucleic acid molecule is that it has a blocked 3′ terminal side and thus unavailable for mispriming or primer-dimer. Linear conventional primers have free 3′ ends that can be bound by partially complementary sequences possibly leading to the 3′ extension by DNA Polymerase and giving rise to false signal or, at least, the reduction/exhaustion of enzymes and co-factors needed for the targeted reaction. This possibility increases exponentially with the number of primers included in a reaction (multiplexing). This limits the multiplexing capability of conventional primers or any primers having accessible 3′ ends. Multiplexing, defined as multiple tests being performed simultaneously in one reaction can be very advantageous especially in clinical diagnostics, research and public health.

An “hairpin” primers is herein referring to a “loopable structures” as known in the art and that's used to effectively avoid the unwanted interactions since any partial complementary sequences from other primers or DNA/RNA sequences will be in competition with their complementary ends. However, this intrinsic complementarity also affects the binding to the intended target making this type of primer less efficient. Relaxing this “mechanically constrained closure”, making it easier to open and close might solve the efficiency of priming.

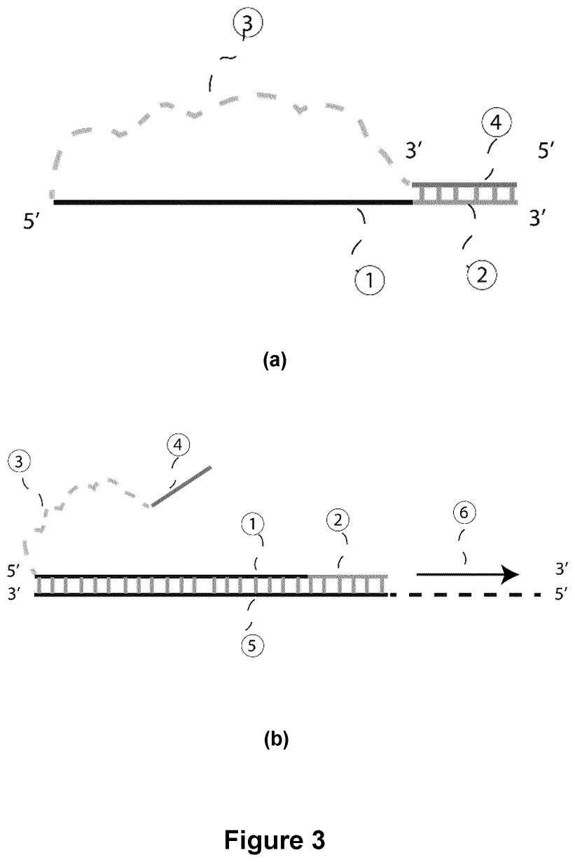

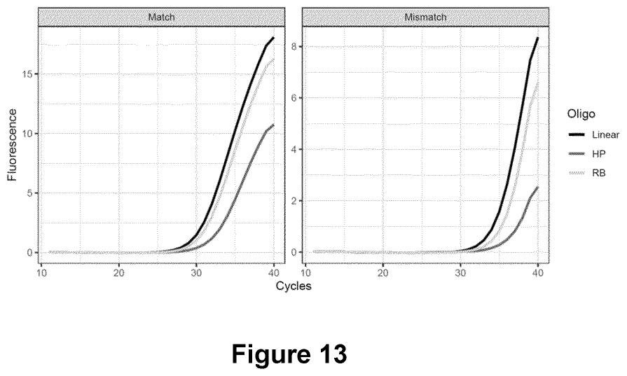

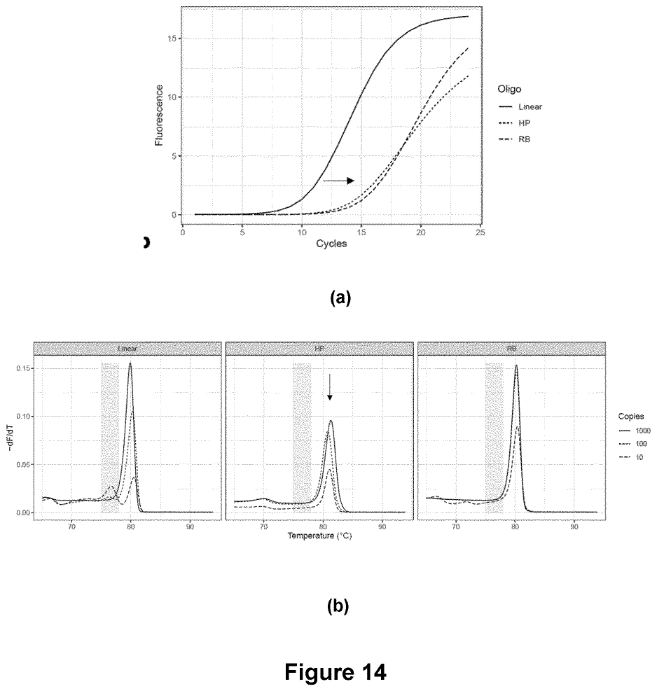

The oligonucleotide nucleic (ReBound primer) of the invention with its linker between the “priming” (1) and “blocking” (4) sequences renders the primer more flexible to open and close leaving the 3′ “hooking region” (2) more available to bind to its target when there is complementarity to the full priming region (1,2) as seen in FIG. 3. This configuration allows the primer to be as efficient as the conventional “linear” primers to anneal and detect even small amounts of target while avoiding partial complementarities as a “hairpin” primer. Since the full priming is as found on a conventional “linear” form of a primer and the open/closure mechanism is more “relaxed”, this primer has the same susceptibility to a mismatch as a “linear” primer as seen in FIG. 13. Something that does not hold true for a “hairpin” primer especially if the mismatch is on the self-complementary region where the primer will more strongly detach and close itself when it encounters even one mismatch. Accordingly, RB Primer with his features has a very high multiplexing capability while retaining high efficiency.

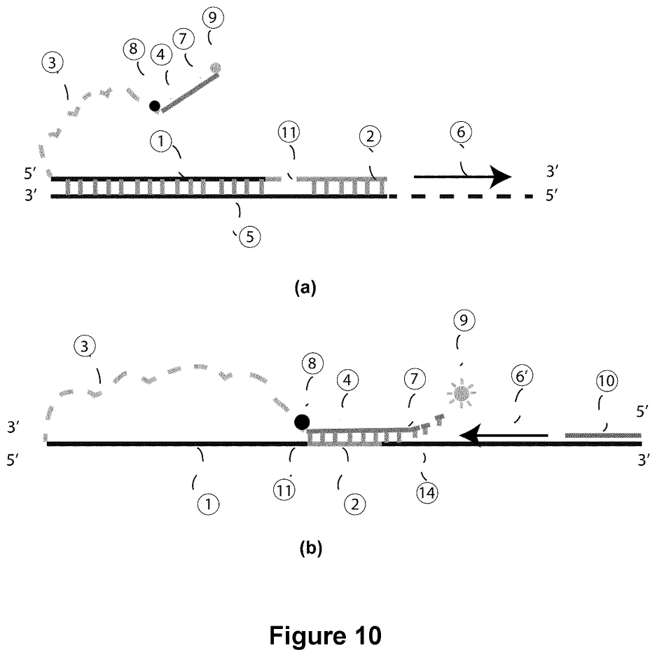

In one preferred embodiment, one modification can be performed on the “RB” primer design allowing for intrinsic fluorescence detection method as portrayed on FIG. 8, where a fluorophore and a non-fluorescent quencher can be incorporated into the blocking region (4). An extended region (7) of the blocking sequence (4), that is not complementary to the hooking sequence (2), can be added to the blocking to achieve the desired binding melting temperature (Tₘ) of the so labelled blocking region to the complementary sequence (14). In fact, the 3′ extension (6) of the bound “hook” region (2) to its target sequence (5) leads to amplification of the sequence. This said sequence (14) complementary to the target (5) can now bind fully to the labelled-blocking region. The extension of the opposite primer (10) 5′ to 3′ will lead to cleavage and release of this label providing the detection of the amplified target as demonstrated in FIG. 10. A blocker (13) should be added if an extension of the blocking (7) is included to avoid that the hooking region (2) is 3′ extended when primer is closed (FIG. 8).

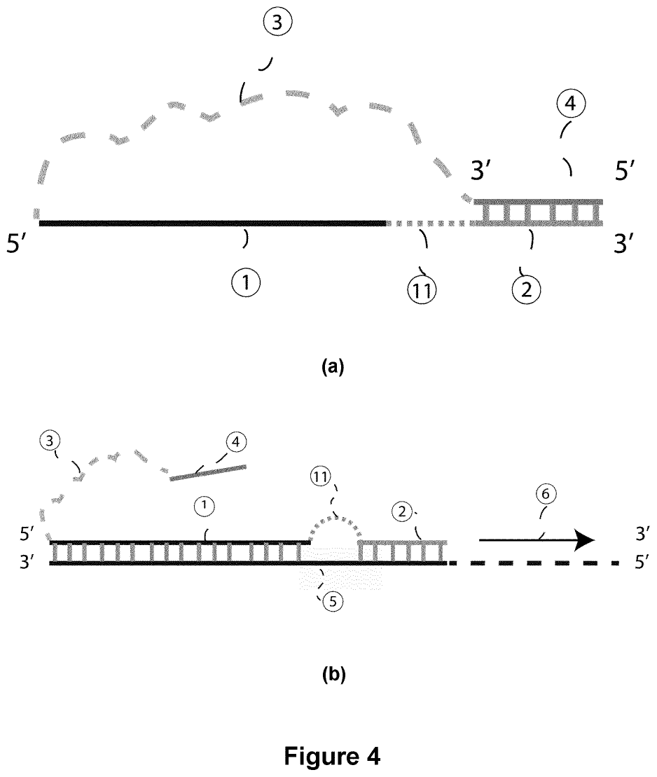

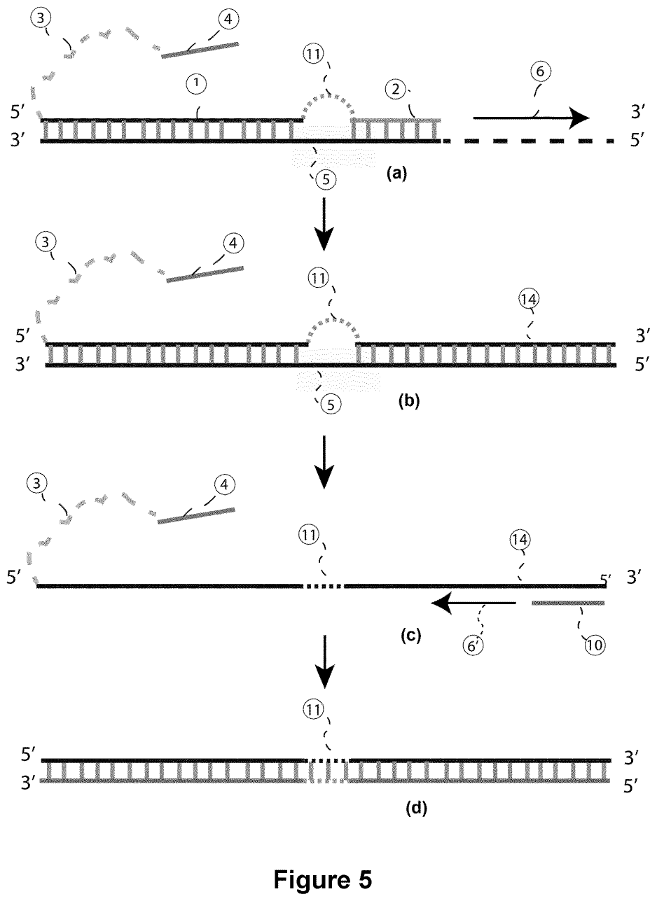

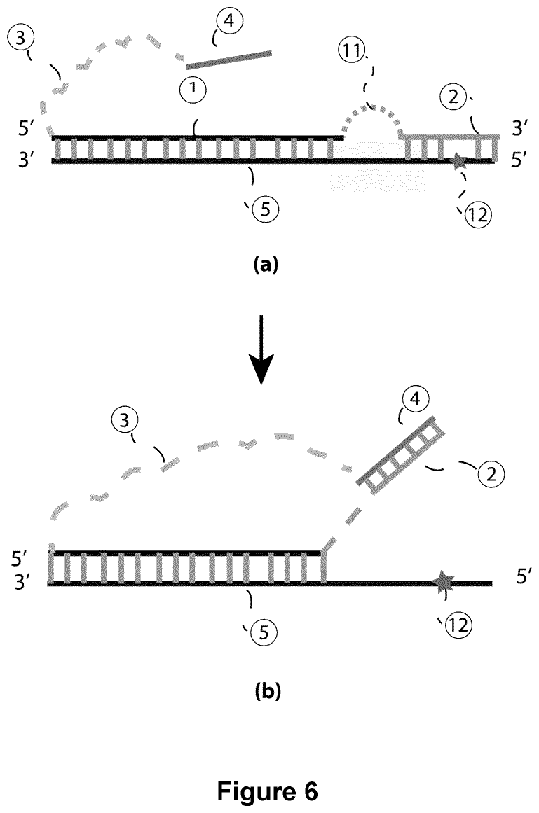

An important feature for diagnostics and always a challenge in regular PCR is to be able to discriminate between very similar targets, including at the single nucleotide level. To be able to achieve this with the flexible RB Primer design, disclosed herein is the inclusion of a spacing region (11) as a sequence segment that is not complementary to the target sequence (5), as displayed in FIG. 4. The addition of this spacing region forms a gap in the priming of this nucleic acid molecule dividing it into strong (1) and weak (2) annealing regions. This feature destabilizes the hooking region rendering it sensitive to mismatches providing selectivity to the RB Primer, being now called Discriminative RB Primer (DRB Primer). In a first step, the DRB primer will encounter the target sequence by the recognition of the longer and stronger priming region (1). This will force the opening of the blocking (4) and hooking regions (2). If the hooking region is a perfect match to the target, binding will occur. In this situation and in the presence of a DNA Polymerase or a Reverse Transcriptase, the 3′ terminus side would be extended in an amplification reaction (14). If even one mismatch (12) is encountered, hooking region (2) would be destabilized and concealed by the blocking region (4), avoiding the binding to the target and eventual amplification and the primer detaches as displayed in FIG. 6b. As the spacing region will be subsequently incorporated in future amplification cycles, the priming region (1,11, 2) will become so strong that the assay sensitivity increases dramatically leaving the discrimination to occur within the first cycles.

The selectivity of the DRB Primer can be fine-tuned by the length and/or strength of annealing (melt temperature, Tm) of each portion: priming (1), hooking (2) and blocking (4). The priming region (1) should have a Tm that is approximate or slightly above the working temperature (for instance PCR annealing temperature). The priming should not have a very high Tm in comparison to the working temperature to not bind strongly and stabilize the primer rendering it less selective. The strength of the “blocking effect” can be assessed by the melting temperature of the ReBound primer

$$( {T}_{m}^{RB} ) .$$

The ReBound primer melting temperature is preferably set to be within the range of ±5° C. of the working temperature (annealing temperature) since the linker provides flexibility to the design and allows the RB primer structure to open if the full priming region is available to bind to the its target sequence. Furthermore, the linker according to the invention, allows to maintain the hooking region (2) sequence with the 3′ terminus in close proximity (without the necessity of being in a closed form) with the blocking region sequence (4), competing thereby with potential undesirable interactions of the 3′ terminus of the hook with other nucleic acid molecules in the same reaction

The hooking region (2) is selective since it is a short sequence. Its selectivity can be further improved by the addition of LNA at the mismatch site as disclosed by Owczarzy et al. (Biochemistry 2011, 50, 9352-9367). Adding LNA to the mismatch site as well as around it (one on the right and one on the left of it) can destabilize the mismatch making the hooking region (2) completely detach. This configuration of the DRB primer with Spacing region (11) and LNAs on the hooking region (2) is highly discriminatory but still sequence dependent. One can choose to add more or less LNAs to adjust the level of discrimination and the cost of the primer. Furthermore, as stated by Owczarzy et al. the nature of the LNA (A, T, G, C) in both mismatch site and surroundings can influence the stability of the mismatch thus it is important to be aware of that and design accordingly. If an LNA should not be placed on the mutation site or surroundings, one can make the blocking region (4) stronger to render the primer more selective. It is this flexibility of the design that poses an advantage to this disclosure in relation to other propositions that are very sequence dependent.

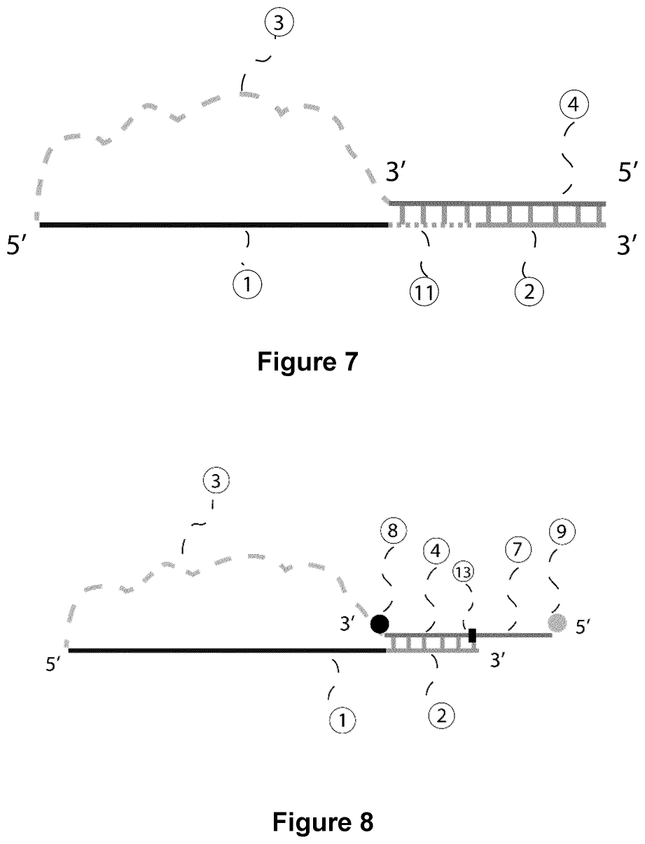

In one preferred embodiment, the blocking region (4) might be complementary not only to the hooking region (2) but also to the spacing region (11), partly or fully, as portrayed in FIG. 7. This provides a double effect: stronger blocking and closer proximity for the blocking sequence in relation to the remaining molecule. This design leaves the blocking region more prone to “capture” the hooking region whenever there is a mismatched template.

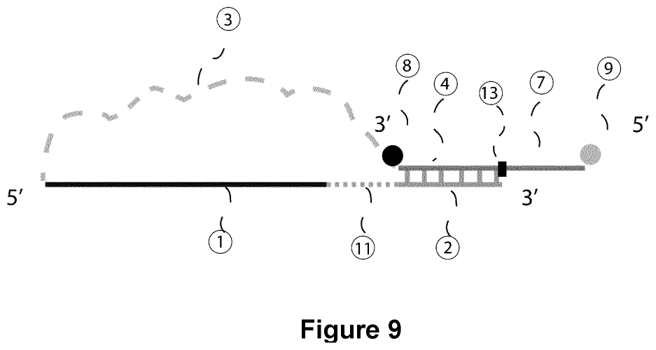

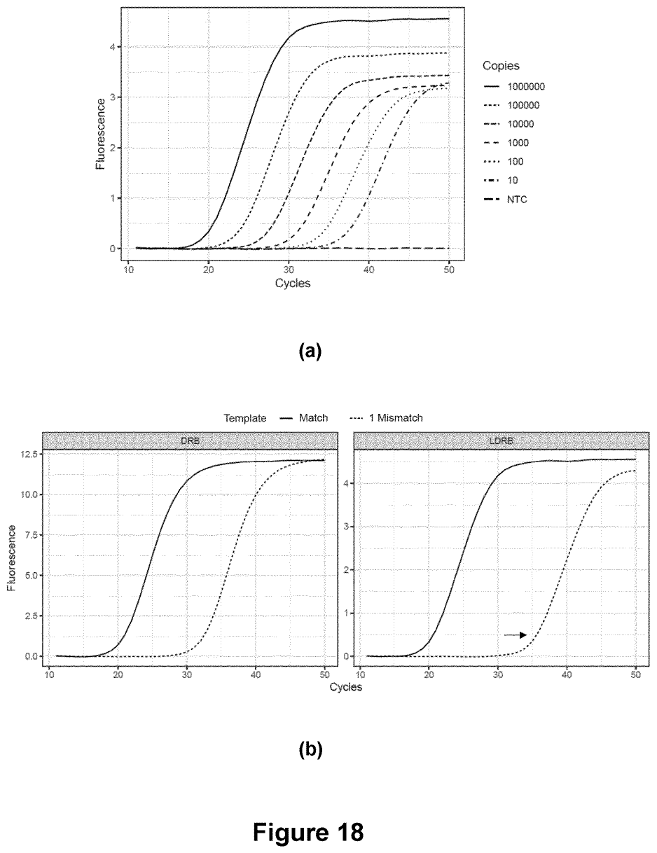

Further modifications to the blocking region can provide other features to the DRB primer, including as stated above the incorporation of fluorophore and quencher on the blocking region for an intrinsic fluorescent detection method (“Labelled DRB Primer, LDRB”, FIG. 9). Importantly, one of the finding of the current disclosure is the fact of including the fluorophore and the quencher in the primer makes increases the discrimination of the primer toward mismatches as shown in FIG. 18.

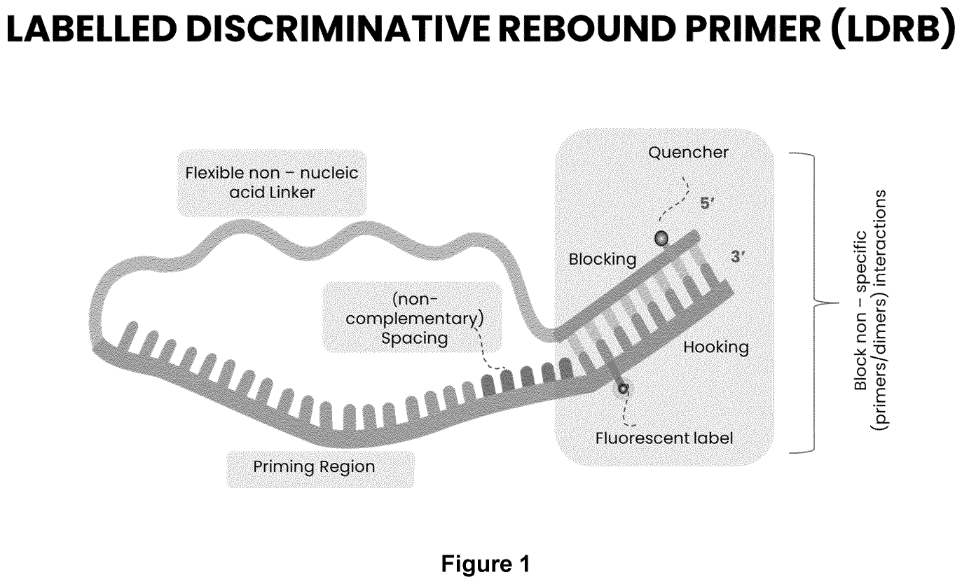

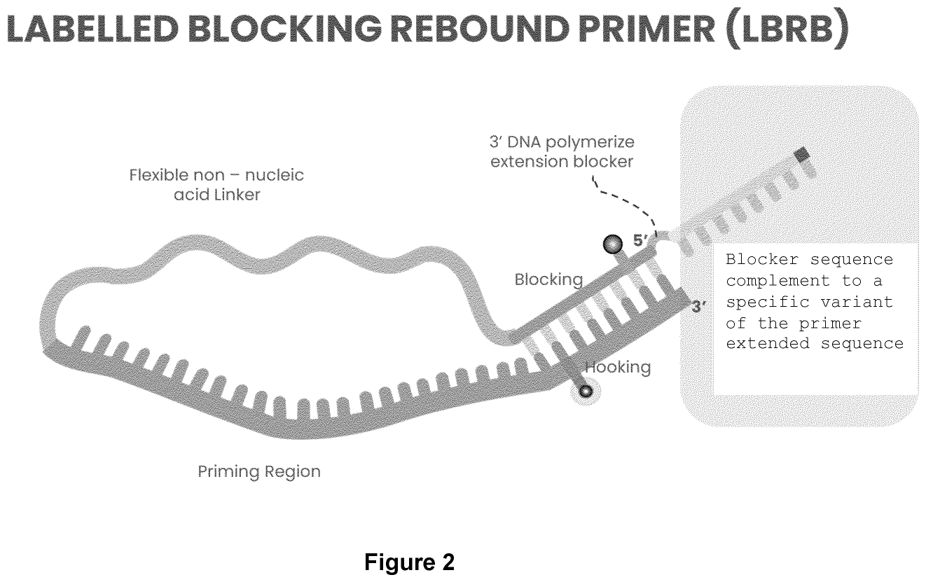

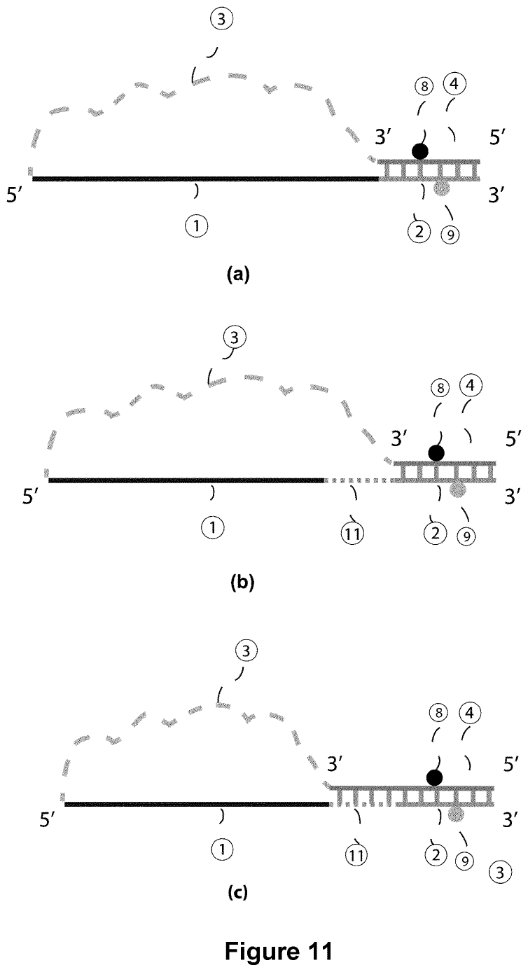

Alternatively, the label and respective quencher might be added in opposing positions as in the hooking and blocking regions, as depicted in FIGS. 1 and 11. The inclusion of a label (9) in the hooking region allows for its incorporation in the extended sequence, signalling it. The fluorescent quencher (8) present in the blocking region (4) will quench the signal whenever it binds to its complementary hooking region (2). This design allows for the analysis of the unused oligos as well as the obtained amplicons at the end of a polymerase-chain reaction by a melting curve analysis. Increasing the temperature of the reaction separates the hooking (2) and blocking (4) regions leading to a fluorescent signal. Lower temperatures, on the other hand, display low signal due to the proximity of the quencher (8) and fluorescent label (9) by the binding of the blocking and hooking regions.

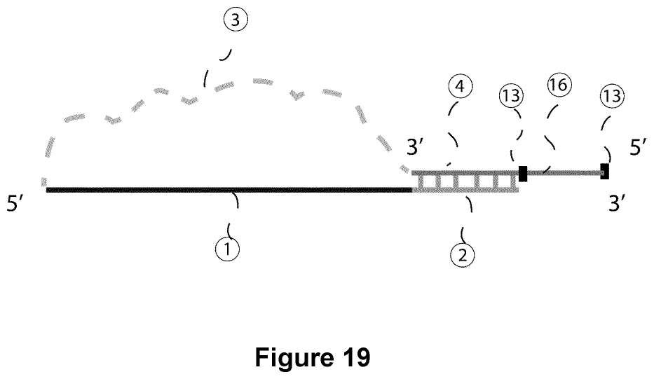

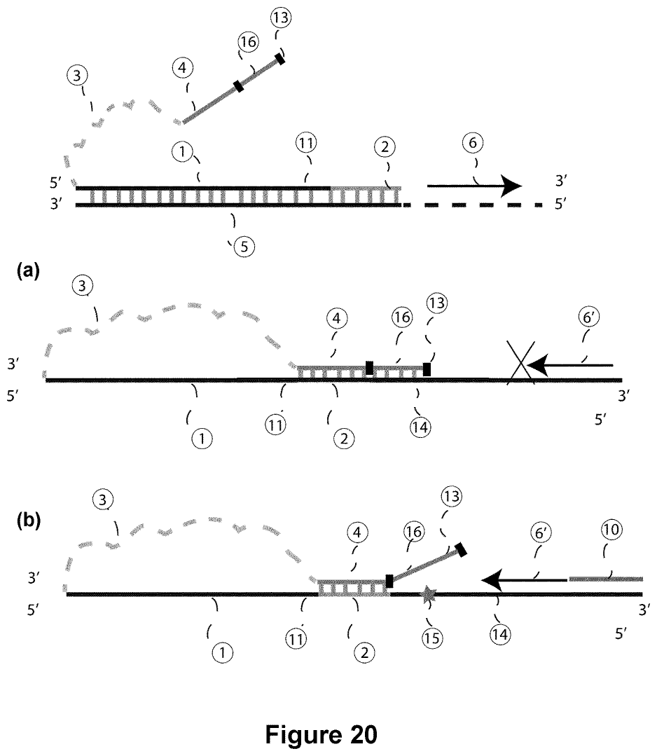

Another addition as disclosed is the incorporation of an extension (16) of the blocking sequence (4) that is not complementary to the hooking sequence (2) and contains an amplification Blocker (13) at the 5′ terminus, as depicted in FIG. 19. The 3′ extension (6) of the bound hooking region (2) to its target sequence (5) leads to amplification of the sequence. This said amplified sequence (14) complementary to the target (5) can now bind fully to the blocking region containing the amplification Blocker (13). The extension of the opposite primer (10) 5′ to 3′ will be halted leading to no amplification of this sequence as shown in FIG. 20. This “Blocking ReBound” Primer embodiment can prove useful when larger insertions or deletions are the origin of the sequence variation to detect. Binding and blocking a portion that is only present in one variant would allow the selection of the other variant (FIG. 20).

### EXAMPLES

### Example 1: Linearity and Efficiency of “RB” Primer in Comparison with Conventional “Linear” and “Hairpin” Primers

A comparison was performed using a dilution series of Template RNA (1000, 100, 10 copies and No template control—NTC) in 3 separate reaction mixtures using either conventional “linear”, “hairpin” or “RB” primers to generate a double-stranded amplification product of 115 nucleotides long. The primer sequences were as follows:

Linear_F:
(SEQ ID No. 1)
TCAAACTGTCAAACCCGGTAA
Linear_R:
(SEQ ID No. 2)
CATCCTGAGCAAAGAAGAAGTGT
Hairpin_F:
(SEQ ID No. 3)
TTACCGTCAAACTGTCAAACCCGGTAA
Hairpin_R:
(SEQ ID No. 4)
ACACTTCATCCTGAGCAAAGAAGAAGTGT
RB_F:
(SEQ ID No. 5)
TTACCGXXXTCAAACTGTCAAACCCGG+TAA
RB_R:
(SEQ ID No. 6)
ACACTTXXXCATCCTGAGCAAAGAAGAAGTGT
Probe:
(SEQ ID NO. 7)
Texas Red-A+GACTTCTATGA+CTTTGCTGT+GTCT-BHQ2

In the nucleotide sequences of the primers, the underlined part represents the additional 5′ complementary sequence to the 3′ segment/hooking region. The X represents a Spacer 18 (HEG). Plus (+) symbol represents LNA modified nucleotide. Using Integrated DNA Technologies' OligoAnalyzer Tool for calculating the melting temperatures of DNA hybrids (qPCR parameter sets: [oligo]=0.3 μM; [Na+]=50 M; [Mg 2+]=3 mM; [dNTPs]=0.8 mM), the calculated Tm of the priming regions were between 62-63° C. Using “Unified Nucleic Acid Folding and hybridization package-UNAFold” available online.

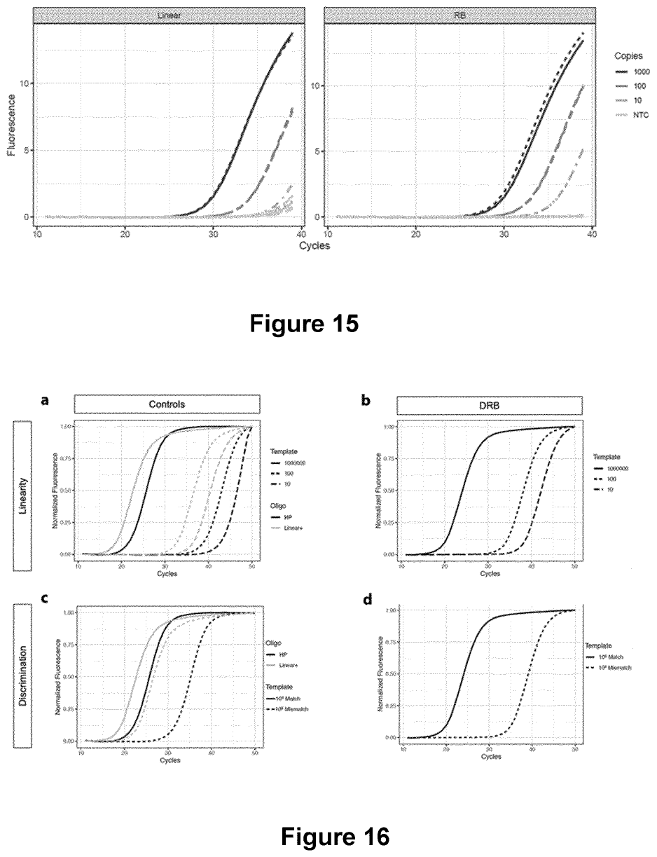

PCR amplifications were performed in a 20-μL reaction volume containing 1× One-Step Takyon Ultra Probe Master Mix (Eurogentec), 300 μM of each of the primers and 125 μM of the probe. Amplifications were carried out in Magnetic Induction Cycler (MIC qPCR Cycler, Biomolecular Systems) using the appropriate MIC tubes and caps. The thermal-cycler profile was 5 min at 50° C., 3 min at 95° C., followed by 40 cycles of 95° C. 5 s and 55° C. for 30 s. Real-time fluorescent results which translate into an increase fluorescent signal intensity as a function of the number of cycles completed and is proportional to the number of copies amplified is shown in FIG. 15. The main issue with using hairpin primers ergo complementary sequences on the extremities of the primers, is the self-complementarity that does not translate into efficient priming. In FIG. 15a, we can see the “hairpin” primers amplify the same number of copies of template later (indicated by the horizontal arrow) which means they are less efficient in providing detectable signal for the same number of copies losing performance for lower amounts of template (10 copies). “RB” primers, however, retain the same linearity and performance as the conventional primer. The assay instrument automatically calculates the threshold cycle (Ct value) for each reaction. In FIG. 15b, we show the corresponding Ct values found on graphs displayed in FIG. 15a. A difference of 2 Ct values was seen for the “hairpin” Primers for the 1000 copies while a difference of 4 Ct values.

### Example 2: Mismatch Sensitivity of the “RB” Primer in Comparison with Conventional “Linear” and “Hairpin” Primers

A PCR amplification and detection assay was performed using the same template (1000 copies) of the RNA and conditions as indicated in Example 1. Only difference was a mismatch (bold underlined) introduced at the priming sequence of the forward primer:

Linear_F:
(SEQ ID No. 8)
TCAAACTGTCAAACCCGGAAA
Hairpin_F:
(SEQ ID No. 9)
TTACCGTCAAACTGTCAAACCCGGAAA
RB_F:
(SEQ ID NO. 10)
TTACCGXXXTCAAACTGTCAAACCCGGAAA

PCR conditions and remaining primers and probe were used as described in Example 1. As seen in FIG. 16, this mismatch affected the detection of the template in a similar manner as the conventional “linear” primer. This shows that this “RB” design is not more discriminative than a conventional primer. This is an important feature in an assay where the target might have some sequence variations, such as pathogens (viruses) whose mutation rate is very high.

### Example 3: Ability to Avoid Unwanted Interactions of the “RB” Primer in Comparison with Conventional “Linear” and “Hairpin” Primers

A dimer containing a 6 nucleotides-long segment at the 3′ region complementary to our primers (SEQ ID No. 11, bold nucleotides) was made to test the ability of the primers (SEQ ID No.1, 3, 5) to avoid this partial complementarity. Conditions were set to favour this interaction to challenge this ability such as: long stretch of partial complementarity with strong standard free energy (ΔG −10.93 Kcal/mol), the targeted template (full complementarity) was not added to the reaction therefore the primers have no other interaction available, low annealing temperature and long extension time (50° C. 1 min) and universal non-selective intercalating dye (EvaGreen®, Biotium) for detection.

Dimer:
(SEQ ID NO. 11)
GATCCGTGGACCGACTAGATGCGTTTACCG

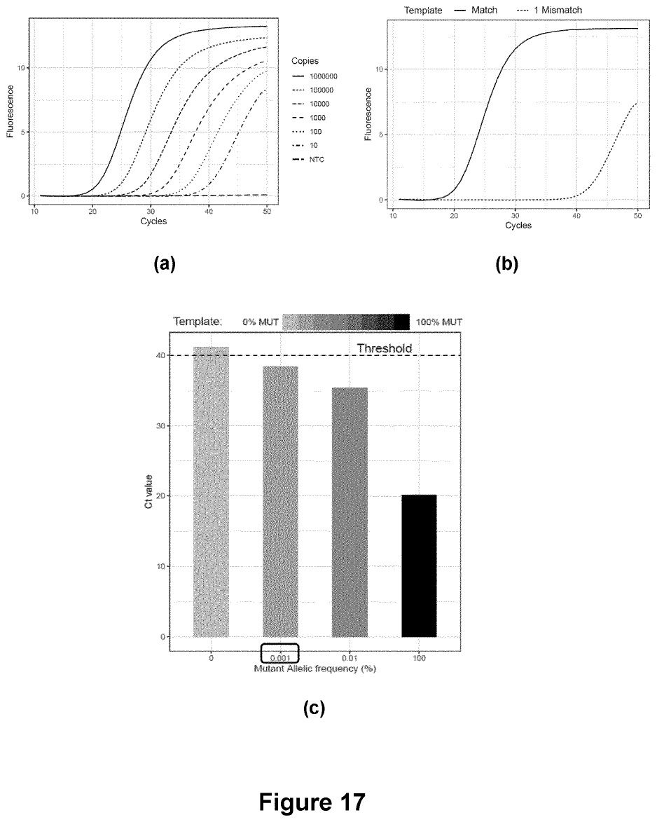

PCR amplifications were performed in a 20-μL reaction volume containing 1× Omnitaq2 containing Master Mix (DNA Polymerase), 200 μM of each the four deoxyribonucleoside triphosphates (dNTPs), 300 μM of each of the primers and dimer, 1× EvagGreen® (Biotium). Amplifications were carried out in Magnetic Induction Cycler (MIC qPCR Cycler, Biomolecular Systems) using the appropriate MIC tubes and caps. The thermal-cycler profile was 2 min at 95° C., followed by 40 cycles of 95° C. 5 s and 50° C. for 1 min. Real-time EvaGreen® fluorescent signal intensity increases as a function of the number of cycles completed and is proportional to the number of copies amplified is shown in FIG. 17a. Placing primer and pre-made dimer together in a reaction without any other template forces the dimers to react and possibly be amplified into a detectable signal. The delay in amplification seen (horizontal arrow) with both “hairpin” and “RB” primers demonstrates the resistance of this “closed” primer design to form cross-dimers in comparison to the conventional “linear” primers. The delay in Ct value is of 4.6 which translates into about 16× less dimers formed.

Sometimes primers contain partial complementary segments within their own design possibly forming self-dimers in a reaction. While these might not be detected when using specific probes, their presence might lead to inefficient PCR and/or exhaustion of cofactors and enzymes. To evaluate this with our tested primer designs, we repeated the same experiment as seen in Example 1 but with EvaGreen® fluorescent dye instead of the specific probe (SEQ ID No. 7). PCR amplifications were performed in a 20-μL reaction volume containing 1× Air-dryable RT-qPCR Master Mix (Meridian Bioscience), 300 μM of each of the primers and 1× EvagGreen® (Biotium). Amplifications were carried out in Magnetic Induction Cycler (MIC qPCR Cycler, Biomolecular Systems) using the appropriate MIC tubes and caps. The thermal-cycler profile was 5 min at 50° C., 2 min at 95° C., followed by 40 cycles of 95° C. 5 s and 55° C. for 30 s followed by melt curve analysis (65° C. to 95° C. at 0.3° C./s). Resulting melting curves are shown in FIG. 17b where “linear” conventional primers fail to efficiently detect low copy number (10 copies), losing performance, as seen in a double small peak comprising both dimers and amplicons. On the other hand, both “hairpin” and “RB” primers managed to avoid the dimers as demonstrated by a single peak corresponding to the amplicons. Importantly, and as stated before, the delay in amplification seen in FIG. 15a for the “hairpin” primers is seen here by a decrease in end-point fluorescent translating into a smaller peak for each amplicon (vertical arrow) on the melting curve analysis.

### Example 4: Multiplexing with “RB” Primers

Within a multiplexing reaction working without any false signals, seen as detectable fluorescent signals on No Template Controls (NTC) and with PCR linearity and efficiency as seen in singleplex for each target, we carried out an addition with the primers and samples mentioned above in Example 1 (SEQ ID No. 1, 2, 5, 6, 7).

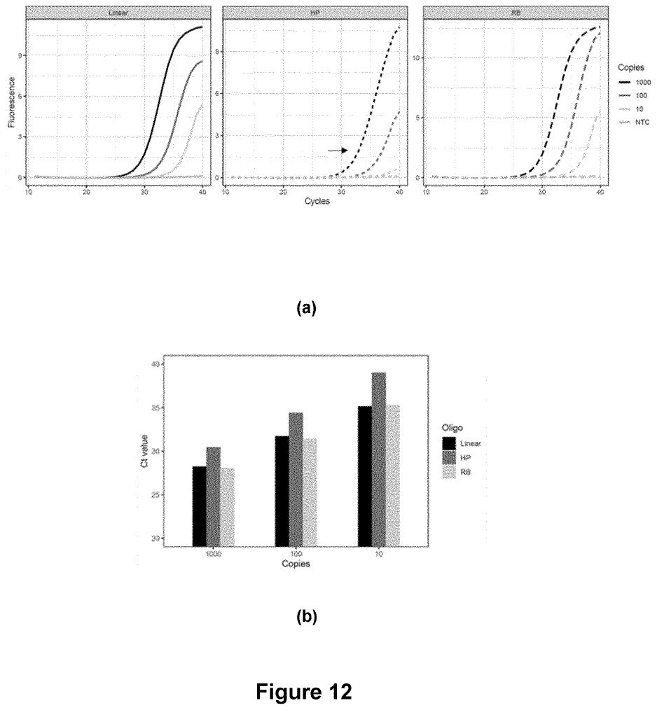

PCR amplifications were performed in a 20-μL reaction volume containing 1× One-Step Takyon Ultra Probe Master Mix (Eurogentec), 150-300 μM of each of the primers and 100-125 μM of the probe fo the different targets. Amplifications were carried out in Magnetic Induction Cycler (MIC qPCR Cycler, Biomolecular Systems) using the appropriate MIC tubes and caps. The thermal-cycler profile was 5 min at 50° C., 3 min at 95° C., followed by 40 cycles of 95° C. 5 s and 60° C. for 30 s. Real-time fluorescent results which translate into an increase fluorescent signal intensity as a function of the number of cycles completed and is proportional to the number of copies amplified is shown in FIG. 15. Adding this one extra set of primers and probe in “Linear” form (SEQ ID No. 1, 2, 7) to an already multiplexed reaction containing 8 linear primers and 4 TaqMan probes resulted in false positive signal masking the result seen for the low copy number (10 copies, FIG. 12). We identified in silico the most likely problematic combinations of primers according to partial complementarity of their sequences (data not shown). Placing only 4 primers into an “RB” design abrogated the false signal. Since it is known that avoiding certain interactions, others might be formed, it is best to place all primers into an “RB” design in a multiplexing reaction to be certain to not have non-specific amplification and signal.

### Example 5: Discrimination and PCR Performance of the “DRB” Primer in Comparison to Conventional “Linear” and “Hairpin” Primers

PCR amplification and detection assays were carried out using either a BRAF WT or mutant BRAF V600E designed 1000 nucleotide-long DNA fragment template (GeneArt, Thermo Fisher Scientific), therefore differing by one single nucleotide. Conventional forward and reverse primers (“linear”) matching the BRAF V600E sequence were used to generate a double-stranded amplification product of 110 nucleotides long using either WT or mutated template. Comparison was made to “hairpin” and “DRB” primers in both discrimination of the single nucleotide difference and PCR performance. The primer sequences were as follows:

Linear_BRAF_F:
(SEQ ID No. 12)
GTGATTTTGGTCTAGCTACAG+AG
Linear_BRAF_R:
(SEQ ID No. 13)
ATAGCCTCAATTCTTACCATCC
HP_BRAF_F:
(SEQ ID No. 14)
CTCTGTAGAA+TAGGTG+A+TTTTGGTCTAGCTACAG+AG
HP_BRAF_R:
(SEQ ID No. 15)
TGGATGGATAGC+CTCAATTCTTACCATCCA
DRB_BRAF_F:
(SEQ ID No. 16)
CTCTGTAGAATTTXXXXGTAA+A+AATA+G+GTGAT
TTTGGAAATTCTACAG+AG
RB_BRAF_R:
(SEQ ID No. 17)
TGGATGGXXXAATAGCCT+CAATTCTTACCATCCA
BRAF_Probe:
(SEQ ID NO. 18)
FAM-ATGGAGTGGGTCCCATCAGTTTGAA-BHQ1

In the nucleotide sequences of the primers, the underlined part represents the additional 5′ complementary sequence to the 3′ segment/hooking region. The X represents a Spacer 18 (HEG). Plus (+) symbol represents LNA modified nucleotide. Bold nucleotide represents the mutation site thus the single nucleotide difference between BRAF WT and BRAF V600E. Using Integrated DNA Technologies' OligoAnalyzer Tool for calculating the melting temperatures of DNA hybrids (qPCR parameter sets: [oligo]=0.2 μM; [Na+]=50 M; [Mg 2+]=3 mM; [dNTPs]=0.8 mM), the calculated Tm of the priming regions of the Forward primers were set at 60° C. Using “Unified Nucleic Acid Folding and hybridization package-UNAFold” available online.

PCR amplifications were performed in a 20-μL reaction volume containing 1× One-Step Takyon Ultra Probe Master Mix (Eurogentec), 250 μM of each of the primers and 125 μM of the probe. Amplifications were carried out in Magnetic Induction Cycler (MIC qPCR Cycler, Biomolecular Systems) using the appropriate MIC tubes and caps as well as CFX96 C1000 PCR Instrument (Biorad). The thermal-cycler profile was 3 min at 95° C., followed by 40 cycles of 95° C. 5 s and 60° C. for 30 s. Real-time fluorescent results which translate into an increase fluorescent signal intensity as a function of the number of cycles completed and is proportional to the number of copies amplified is shown in FIG. 18 for all tested primers design. One LNA was introduced at the mutation site knowing that the mismatch would be more destabilized (Owczarzy et al., Biochemistry 2011, 50, 9352-9367) and then primer designs were compared.

FIG. 18 displays the results of the PCR amplification using 10⁶, 100 and 10 copies of the BRAF V600E template and the conventional “linear” and “hairpin” primers (controls) to check performance and linearity in comparison to “DRB” Primer (a, b). While we can see that the hairpin primer shows later amplification for the higher quantity of template tested (10⁶ copies), the delay of amplification for the lower copy numbers is exacerbated, proving this design very inefficient (a). On the other hand, “DRB” portraits similar amplification results as the “linear” primer translating into comparable linearity and performance (b).

When the amplification of 10⁶ copies of mutant BRAF V600E template are compared to 10⁶ copies of BRAF WT template (one mismatch), “hairpin” primer presented a higher delay hence discrimination in comparison to the “linear” form of the primer (c) with a Ct difference of 9 values as opposed to the 3 Ct value delay for a mismatched template that was seen with the “linear” primer. An improvement in discrimination is shown in FIG. 19d with DRB primer with a 15 Ct value delay.

### Example 6: Fine-Tuning Discrimination Using “DRB” Design

PCR amplification and detection was carried out as described above in Example 5 using different forward DRB primer to add discriminatory power to the design. PCR amplifications were performed in a 20-μL reaction volume containing 1× One-Step Takyon Ultra Probe Master Mix (Eurogentec), 250 μM of each of the primers, 125 μM of the probe and 20 mM of tetramethylammonium chloride (TMAC). Amplifications were carried out in Magnetic Induction Cycler (MIC qPCR Cycler, Biomolecular Systems) using the appropriate MIC tubes and caps. The thermal-cycler profile was 3 min at 95° C., followed by 40 cycles of 95° C. 5 s and 60° C. for 30 s. The forward primer tested was as follows:

DRB2_BRAF_F:
(SEQ ID NO. 19)
+CTCTGTAGXXXXCA+CAGTAA+A+AATA+G+GTGAT
TTTGGAGATCCTACA+G+AG

In the nucleotide sequences of the primer, the underlined part represents the additional 5′ complementary sequence to the 3′ segment/hooking region. The X represents a Spacer 18 (HEG). Plus (+) symbol represents LNA modified nucleotide. Bold nucleotide represents the mutation site thus the single nucleotide difference between BRAF WT and BRAF V600E. Using Integrated DNA Technologies' OligoAnalyzer Tool for calculating the melting temperatures of DNA hybrids (qPCR parameter sets: [oligo]=0.2 μM; [Na+]=50 M; [Mg 2+]=3 mM; [dNTPs]=0.8 mM), the calculated Tm of the priming regions of the Forward primer was 67° C. and the calculated Tm_{Hairpin} was 65° C. Overall, a high Tm priming region, double LNA hooking and a strong Tm of the hairpin translating into a strong blocking region allows for a more selective hooking region and a robust primer with high PCR efficiency, as seen in FIG. 20. A dilution series of the BRAF V600E mutant template from 10⁶ to 10 copies using this DRB primer (SEQ ID No. 19) demonstrated around 90% PCR efficiency (a). One single mismatch of BRAF WT template (10⁶ copies) led to 22 Ct value delay (b) in comparison with similar copy number of matched 10⁶ copies BRAF V600E template, proving this primer design very selective. In FIG. 20c are plotted the detected Ct values for the diluted BRAF V600E template in a mismatched BRAF WT template background of 10⁶ copies, where 0 copies of the mutant indicate the presence of only the WT background. Differentiating the detection of the low copy number (10 copies) of the BRAF V600E from the BRAF WT template sets the sensitivity at 0.001%.

### Example 7: Testing a Labelled DRB Primer (LDRB)

A different forward DRB primer and corresponding design with fluorophore and quencher on the blocking region was subsequently tested. Primer sequences are as follows:

DRB3_BRAF_F:
(SEQ ID No. 20)
+CT+CTGTAGXXXXCA+CAG+T+AAAAATA+G+GTGATTTTG
GAGATCCTACA+G+AG
LDRB3_BRAF F:
(SEQ ID NO. 21)
BHQ1-+CT+CTGTAGXXXXCA+CAG+T+AAAAATA+G+GTGATT
TTGGAGATCCT(FAM)ACA+G+AG

In the nucleotide sequences of the primer, the underlined part represents the additional 5′ complementary sequence to the 3′ segment/hooking region. The X represents a Spacer 18 (HEG). Plus (+) symbol represents LNA modified nucleotide. Bold nucleotide represents the mutation site thus the single nucleotide difference between BRAF WT and BRAF V600E. Using Integrated DNA Technologies' OligoAnalyzer Tool for calculating the melting temperatures of DNA hybrids (qPCR parameter sets: [oligo]=0.2 μM; [Na+]=50 M; [Mg 2+]=3 mM; [dNTPs]=0.8 mM), the calculated Tm of the priming regions of the Forward primer was 69° C. and the calculated Tm_{Hairpin} was 67° C. For the PCR amplification and detection same conditions as described above on Example 5 were carried out, except for the reaction testing the LDRB where the probe was omitted, and the concentration of this primer was lowered to 0.2 μM.

Results for this test are shown in FIG. 18 demonstrating the efficiency of the LDRB design to concomitantly amplify and detect linearly a 10-fold dilution of the matched BRAF V600E template without providing signal to a no template control—NTC (a). Comparing the design with and without the fluorophore and the quencher on the blocking region (b), the LDRB adds about 4 Ct value difference (horizontal arrow) to the discrimination between BRAF V600E and BRAF WT template (one mismatch) in comparison to the already discriminating 11 Ct value seen with the same DRB design without the fluorescent label.

Those skilled in the art will appreciate that various adaptations and modifications of the just-described preferred embodiments can be configured without departing from the scope and spirit of the invention. Therefore, it is to be understood that, within the scope of the appended claims, the invention may be practiced otherwise than as specifically described herein.

1. An oligonucleotide nucleic acid molecule for binding to a region of a target nucleic acid comprising:
(a) a first single-stranded nucleic acid sequence, having a 5′ terminus and a 3′ terminus, and being complementary at least in part to the target sequence (5);
wherein the said complementary portion comprises a priming region (1) and a hooking region (2) positioned respectively at the 5′ and 3′ terminus sides of the said first single-stranded nucleic acid sequence.
(b) a second blocking single-stranded nucleic acid sequence (4), having a 5′ terminus and a 3′ terminus, which is at least in portion complementary to the hooking region of the first single-stranded nucleic acid sequence.
(c) a linker region (3) connecting the 5′ terminus of the first single-stranded nucleic acid sequence to the 3′ terminus of the second single-stranded nucleic acid sequence, wherein the linker comprises at least one non-nucleic acid chemical group.

2. An oligonucleotide nucleic acid molecule for binding to a region of a target nucleic acid of claim 1, wherein the length of the priming region (1) is between 15 to 40 nucleotides.

3. An oligonucleotide nucleic acid molecule for binding to a region of a target nucleic acid of claim 1, wherein the length of the hooking region (2) is between 4 to 15 nucleotides.

4. An oligonucleotide nucleic acid molecule for binding to a region of a target nucleic acid of claim 1, wherein the linker (3) is composed from Polyethylene glycol.

5. An oligonucleotide nucleic acid molecule for binding to a region of a target nucleic acid of claim 5, wherein Polyethylene glycol is poly (hexaethylene glycol) H(OCH₂CH₂)₆ₙOH linker, with n varies between 1 and 18 and preferably between 3 and 12.

6. An oligonucleotide nucleic acid molecule for binding to a region of a target nucleic acid of claim 1, which further comprises a label.

7. An oligonucleotide nucleic acid molecule for binding to a region of a target nucleic acid of claim 6, wherein the label is a fluorescence resonance energy transfer (FRET) label system that includes a fluorescent reporter molecule (9) and a quencher molecule (8).

8. An oligonucleotide nucleic acid molecule for binding to a region of a target nucleic acid of claim 7, wherein the fluorescent reporter molecule (9) and a quencher molecule (8) are located on the blocking single-stranded nucleic acid sequence (4).

9. An oligonucleotide nucleic acid molecule for binding to a region of a target nucleic acid of claim 7, wherein the fluorescent reporter molecule (9) and a quencher molecule (8) are separately located on the blocking single-stranded nucleic acid sequence (4) and the hooking region (2).

10. An oligonucleotide nucleic acid molecule for binding to a region of a target nucleic acid of claim 1, wherein the first single-stranded nucleic acid sequence further comprises a spacing region (11), with no complementarity to the target sequence, located between the priming (1) and the hooking (2) regions.

11. An oligonucleotide nucleic acid molecule for binding to a region of a target nucleic acid of claim 10, wherein the spacing region (11) is between 5-10 nucleotides.

12. An oligonucleotide nucleic acid molecule for binding to a region of a target nucleic acid of claim 11, wherein the blocking single-stranded nucleic acid sequence (4) extends to be at least in portion complementary to the spacing region (11).

13. An oligonucleotide nucleic acid molecule for binding to a region of a target nucleic acid of claim 11, wherein the spacing region (11) has a specific sequence used as unique identifier of the said nucleic acid molecule.

14. An oligonucleotide nucleic acid molecule for binding to a region of a target nucleic acid of claim 1, wherein the hooking region (2) is extendable on the 3′ terminus by a sequence polymerization reaction.

15. An oligonucleotide nucleic acid molecule for binding to a region of a target nucleic acid of claim 14, used as a primer in an enzymatic amplification of the target nucleic acid.

16. An oligonucleotide nucleic acid molecule for binding to a region of a target nucleic acid of claim 1, wherein the blocking sequence (4) further comprises an extension sequence (16) positioned at its 5′ terminus characterized by not being: (a) complementary to the hooking region (2), (b) an active template for the extension of the hooking region (2), while bound to the blocking sequence (4), by a sequence polymerization reaction.

17. An oligonucleotide nucleic acid molecule for binding to a region of a target nucleic acid of claim 16, wherein the characteristic (b) is achieved by having a mismatch, positioned at the end of 3′ terminus of the hooking region (2), between the blocking sequence (4) and the hooking region (2).

18. An oligonucleotide nucleic acid molecule for binding to a region of a target nucleic acid of claim 16, wherein the characteristic (b) is achieved by having a sequence polymerization reaction blocker (13) positioned at the 5′ terminus of the blocking sequence (4).

19. An oligonucleotide nucleic acid molecule for binding to a region of a target nucleic acid of claim 16, wherein the extension sequence (16) is designed to be complementary and bind to the antiparallel sequence (14) resulted from the extension of the hooking region (2) upon its binding to the target nucleic acid (5) by a sequence polymerization reaction.

20. An oligonucleotide molecule for detecting a first allelic variant of a target nucleic acid sequence in a sample suspected of comprising at least a second allelic variant of the said target nucleic acid sequence, comprising:
a) a first single-stranded nucleic acid sequence (1, 2), having a 5′ terminus and a 3′ terminus, and being complementary at least in part to the target sequence (5).
b) The said complementary portion of the first single-stranded nucleic acid sequence comprises a priming:
I. a priming region (1), with a length between 15 to 40 nucleotides, located at the 5′ terminus of the said first single-stranded nucleic acid sequence and that bind indiscriminately to both first and second allelic variants.
II. a hooking region (2), with a length between 4 to 15 nucleotides, located at the 3′ terminus of the said first single-stranded nucleic acid sequence and that bind discriminately with a higher affinity to a first allelic variant compared to the second allelic variant.
III. a spacing region sequence (11) which is not complementary to the target sequence (5), having a length between 5 to 10 nucleotides, located between the priming (1) and the hooking (2) regions.
c) a second blocking single-stranded nucleic acid sequence (4), having a 5′ terminus and a 3′ terminus, which is at least in portion complementary to the hooking region of the first single-stranded nucleic acid sequence.
d) a linker region (3) connecting the 5′ terminus of the first single-stranded nucleic acid sequence (1) to the 3′ terminus of the second single-stranded nucleic acid sequence (2), wherein the said linker (3) has no more than five contiguous bases that are complement to first nucleic acid sequence (1,2) or to the target nucleic acid sequence.

21. An oligonucleotide nucleic acid molecule for detecting a first allelic variant of a target nucleic acid sequence of claim 20, which further comprises a label.

22. A method for selectively detecting a first allelic variant of a target nucleic acid sequence in a sample suspected of comprising at least a second allelic variant of the said target nucleic acid sequence, comprising the use of a primer nucleic acid molecule for binding to a region of the said target nucleic acid having a composition of claim 20.

23. A method according to claim 22, wherein the first allelic variant is a mutated variant that comprises single nucleotide substitutions, insertions, or deletion variants.

24. A method for selectively detecting a first allelic variant of a target sequence in a sample suspected of comprising at least a second allelic variant of the said target nucleic acid sequence of claim 23, wherein the hooking region (2) of the primer is perfectly complementary to the first target allelic variant sequence at the mutated nucleotides positions.

25. A method for selectively detecting a first allelic variant of a target sequence of claim 24, wherein the hook region (2) contains at least one modified nucleic acid.

26. A method for selectively detecting a first allelic variant of a target sequence of claim 25, wherein the said modified nucleic acids are Locked Nucleic Acid (LNA).

27. A method for selectively detecting a first allelic variant of a target sequence in a sample suspected of comprising at least a second allelic variant of the said target nucleic acid sequence of claim 1, wherein the said primer has an extension sequence (16) designed to be complementary and bind to the extended antiparallel sequence (14) of the second target allelic variant, blocking thereby the extension of the antiparallel sequence (6′) by a reverse primer (10).

28. A kit for detecting at least one target nucleic acid in a sample comprising one oligonucleotide molecule of claim 1 and claim 21.
